(19) United States
(12) Patent Application Publication
(10) Pub. No.: US 2009/0326384 A1
(43) Pub. Date: Dec. 31, 2009

(54) Device With Integrated Multi-Fiber Optical Probe and Methods of Use

(75) Inventors: Irving Bigio, Chestnut Hill, MA (US); Satish Singh, Sharon, MA (US)

Correspondence Address: RONALD I. EISENSTEIN, 100 SUMMER STREET, NIXON PEABODY LLP, BOSTON, MA, 02110, US

(73) Assignees: Trustees of Boston University, Boston, MA (US); Boston Medical Center Corporation, Boston, MA (US)

(21) Appl. No.: 12/374,175

(22) PCT Filed: Jul. 18, 2007
(86) PCT No.: PCT/US07/16267

§ 371 Date: Jan. 16, 2009

Related U.S. Application Data
(60) Provisional application No. 60/831,699, filed on Jul. 18, 2006.

(51) Int. Cl. A61B 6/00; A61B 1/32

(52) U.S. Cl. 600/476; 600/202

(57) ABSTRACT

Biopsy instruments are integrated with a multi-fiber optical probe adapted to perform diagnostic measurements. In addition to being able to analyze, treat or remove tissue, such integrated devices characterize tissue by measuring the amount of scattering and absorption of light transmitted into the tissue. Each fiberoptic probe has an illuminating fiber that provides a broadband light source for transmission into tissue, and a collecting fiber that collects the light scattered by the tissue and transmits the collected light to a spectrometer. One embodiment is an endoscope-mediated tool with a jaw-type biopsy forceps and a multi-fiber optical probe which is conveyed through a hollow central channel. Another embodiment is an endoscope-mediated tool with a jaw-type biopsy forceps and a plurality of multi-fiber optical probes. Yet another embodiment is an endoscopic polypectomy-type snare catheter with a multi-fiber optical probe located at the tip.

### CROSS REFERENCE TO RELATED APPLICATIONS

This application claims priority to U.S. Provisional Application No. 60/831,699 filed Jul. 18, 2006, the contents of which are incorporated herein by reference.

### GOVERNMENT SUPPORT

The present invention was made with Government Support under Contract No. CA104677 awarded by the National Institutes of Health. The Government has certain rights to the invention.

### BACKGROUND OF INVENTION

1. Field of Invention

The present invention relates generally to instruments for treating or analyzing tissue, and the use of such instruments; more particularly, to devices and methods for treating or analyzing tissue, such as for an optical biopsy, that employ instruments with fiberoptic probes containing multiple optical fibers to implement absorbance-scattering spectroscopies and also being able to simultaneously perform a biopsy. Methods of using such instruments for treatment and diagnosis of diseases, such as cancer are also provided.

2. Description of the Related Art

Optical spectroscopies have become the basis for research activity directed toward the development of novel, non-invasive technologies for tissue diagnostics. Such technologies are classified as “optical biopsy,” although no tissue is actually removed, as with conventional biopsy. A motivation of these technologies is to be able to use such techniques to reduce the need for surgical removal of biopsy tissue samples or to minimize the error rate caused by removing non-pertinent sections of tissue. In optical biopsy, spectral data of the tissue under examination is typically first recorded in vivo with an imaging system or with an optical probe placed on or near the surface of the tissue. Diagnosis of the tissue is then conducted based on the recorded spectral data. In general, optical biopsy technologies provide diagnostic information, in situ, non-invasively, and in real time. Thus, optical diagnostic techniques provide advantages over current methods that present the risks of surgery and delays in diagnosis.

Various spectroscopies have been researched for optical diagnosis. The basic approaches can be used for the detection of certain cancers in addition to other types of diagnosis, such as those that require the measurement of blood oxygen saturation or the identification of some different tissue types.

Biochemical information about a tissue can be obtained by measuring absorption, fluorescence, or Raman scattering signals. To date, much of the work in the area of optical diagnosis has focused primarily on ultraviolet-induced fluorescence spectroscopy, which detects important biochemical changes through the changes they cause in the intrinsic fluorescence spectrum of tissue.

Elastic scattering spectroscopy (ESS) has been employed for tissue diagnosis, where the tissue pathologies are detected and diagnosed using spectral measurements of elastic-scattered light. The recorded spectral data from ESS is sensitive to both the scattering and absorption properties of the tissue for a wide range of wavelengths. The approach of ESS is based on the fact that many tissue pathologies, including a majority of cancer forms, exhibit significant architectural changes at the cellular and sub-cellular level. Therefore, ESS is capable of reporting certain cellular and subcellular architectural features that are a typical part of a pathologist's microscopic assessment and diagnosis.

In particular, ESS spectra relate to the wavelength-dependence and angular-probability of scattering efficiency of tissue micro-structures, in addition to absorption bands. Thus, ESS records spectral signatures that correlate with histological features such as the size/shape of subcellular components, nuclear-to-cytoplasmic ratio, and cell/organelle clustering patterns.

ESS diagnosis can be based either on heuristic models that predict changes in the scattering spectrum corresponding to altered ultrastructure, or on quantitative models that have been used to determine nuclear size in epithelial layers.

Several studies have reported clinical correlations between scattering spectra and mucosal histopathology. For instance, ESS was reported as being applied in vivo in a retrospective clinical study of the urinary bladder where sensitivity and specificity for the detection of malignant tissue was reported to exceed 97%. In addition, ESS has been employed in studies involving the diagnosis of luminal gastrointestinal tract neoplasms. In one study, colorectal ESS measurements were reported as used to develop a spectral metric based on regions of the hemoglobin absorption bands (400-440 nm and 540-580 nm) to identify sites that were dysplastic, adenomatous, and/or cancerous, where the sensitivity was reported to be 100% and the specificity to be 98%. In another study, dysplastic and hyperplastic colonic polyps were reported as distinguished using ESS and neural-network pattern recognition for spectral classification. In addition, ESS has been reported as being applied to the classification of colon polyps. Furthermore, ESS has been reported as being able to identify dysplastic Barrett's esophagus with a sensitivity and specificity of 82% and 80%, respectively in that study.

One study has described ESS applications for breast cancer as (1) an alternative to fine-needle aspiration cytology, and (2) intraoperative assessment of the adequacy of excision margins and of the metastatic status of sentinel lymph nodes. Meanwhile, other groups have discussed further applications of ESS for identifying tissues during invasive procedures. For instance, ESS has been reported as being used to distinguish white from gray matter during probe placement into the brain and to diagnose cervical and ovarian pathologies. However, further understanding of how ESS may be applied is desired. The studies involving ESS demonstrate an increasing need for devices that facilitate further research or that permit more feasible use of ESS in a clinical environment.

### SUMMARY OF THE INVENTION

Embodiments of the present invention are directed to an integrated multi-fiber optical probe adapted to perform diagnostic measurements and a tissue manipulation apparatus, such as a biopsy instrument or a drug delivery or treatment device. Such embodiments enable ESS spectra to be collected and accurately coordinated with tissue treatment and/or diagnosis such as for a biopsy or ablation. Embodiments of the present invention are able to characterize tissue by measuring the amount of scattering and absorption of light transmitted into the tissue and using that information in real time. Each fiberoptic probe has at least two fibers. In particular, each fiberoptic probe has at least one illuminating fiber that provides either discrete wavelengths or a broadband light source, e.g. 320 to 850 nm, for transmission into tissue. In one embodiment, the light source may be a pulsed short-arc lamp. Each fiberoptic probe also has at least one collecting fiber that collects the light scattered by the tissue and transmits the collected light to an analyzing spectrometer or other detector. According to the present invention, the illumination fiber and the collection fiber of the multi-fiber probe can have optical geometries, which specify fiber separation, fiber angle, and fiber facet angle.

In one embodiment of the present invention, the tissue manipulation apparatus is an optical forceps device. The optical forceps device is an endoscope-mediated tool with a jaw-type biopsy forceps and a multi-fiber optical probe which is conveyed through a hollow central channel. The tip of the multi-fiber optical probe, which may contact the examined tissue or illuminate the tissue from a small distance, may be located at, or near, the hinge of the jaws of the forceps. In one variation, the multi-fiber optical probe is designed so that it is extended to make contact with the tissue surface and perform the ESS measurements—a determination can be made at that time whether the tissue being examined should be physically manipulated, e.g., physically removed by forceps or snare. In an alternative variation, the multi-fiber optical probe is fixed and the measurements are performed when the tissue is grasped between the jaws of the forceps. One may use the apparatus to precisely deliver drugs to a specific type of tissue. In addition, the device may be adapted to enable cautery or other types of ablation.

One embodiment of the present invention is a multi-probe optical forceps device. The multi-probe optical forceps device is a tool, to be conveyed through a lumen of an endoscope, with a jaw-type biopsy forceps and a plurality of multi-fiber optical probes. In particular, a multi-fiber optical probe may be located within each of the jaws of the forceps in addition to one located at the hinge of the two jaws. The additional multi-fiber optical probes enable the collection of additional ESS measurements, such as backscatter and forward scatter measurements.

In another embodiment, an optical snare device is encompassed. The optical snare device is an endoscopic polypectomy-type snare catheter with a multi-fiber optical probe located at the tip. In particular, the optical probe is adapted to perform ESS measurements on tissue before the wire-loop of the snare is used to remove a polyp.

In all cases, the integrated multi-fiber optical probe can be made disposable and replaceable to minimize cross-contamination and to facilitate preparation for procedures.

The probes can be used in methods for the detection of disease or disorder in a tissue, particularly a malignancy. In a preferred embodiment, the detection of disease or disorder is in the gastrointestinal (GI) tract or other hollow organ, e.g., the esophagus, stomach, colon, or bladder.

In addition to methods of detection, methods for the simultaneous detection and a physical biopsy and/or focal ablation of a tissue in a single step are encompassed. The devices of the present invention improve upon known techniques by allowing for a more detailed analysis of a tissue, such as depth, and also allow for the real time imaging and then biopsy and/or ablation, thereby decreasing the error rate or trauma common when a tissue is first imaged and then re-entered for biopsy.

In one embodiment, the invention provides a non-invasive real-time method of detecting and/or treating pre-cancer or cancer of a target tissue, comprising the steps of contacting the device with the target tissue, analyzing the output spectra, comparing the output spectra with a training set of spectra or analyzed by a diagnostic algorithm derived from a training set. The training set of spectra can comprise spectra obtained from normal tissue comparable with the diagnostic target, and various disease conditions affecting such tissue. For example, if the target tissue is colon polyps, the training spectra may have one or more spectra from hyperplastic polyps and one or more adenomatous polyps and such. When one compares the target tissue spectrum or spectra with the training set, one looks for as near a match as possible. This can be done visually or by using a variety of types of pattern recognition software. If the tissue is determined to be suspect it can be physically manipulated, e.g., biopsied, treated, etc.

These and other aspects of the present invention will become more apparent from the following detailed description of the preferred embodiments of the present invention when viewed in conjunction with the accompanying drawings.

### BRIEF DESCRIPTION OF THE FIGURES

FIG. 1 illustrates an embodiment of a system that employs a device according to the present invention.

FIG. 2 illustrates an embodiment of the multi-fiber optical probe employed by a device according to the present invention.

FIG. 3A illustrates an embodiment of the present invention employing a biopsy forceps with a multi-fiber optical probe.

FIG. 3B illustrates another view of the embodiment of FIG. 3A.

FIG. 3C illustrates a picture of an embodiment of the present invention employing a biopsy forceps with a multi-fiber optical probe.

FIG. 4 illustrates a further embodiment of the present invention employing a biopsy forceps with a plurality of multi-fiber optical probes.

FIG. 5A illustrates an embodiment of the present invention employing a biopsy snare with a multi-fiber optical probe, where the biopsy snare is disengaged from a polyp.

FIG. 5B illustrates an embodiment of the present invention employing a biopsy snare with a multi-fiber optical probe, where the biopsy snare engages a polyp.

FIG. 5C illustrates a cross-sectional view of a dual lumen central channel which may be employed by an embodiment of the present invention.

FIG. 5D illustrates an embodiment of the present invention employing a biopsy snare with a multi-fiber optical probe, where the biopsy snare is fully deployed from the sheath.

FIG. 5E illustrates an embodiment of FIG. 5D, where the biopsy snare is partially retracted into the sheath.

FIG. 5F illustrates an embodiment of FIG. 5D, where the biopsy snare is fully retracted into the sheath.

FIG. 6 illustrates an example of a detachable connection between a manipulating element and a disposable embodiment of the present invention.

FIG. 7 illustrates an example of a spectra obtained using optically targeted detection of colonic tissue.

### DETAILED DESCRIPTION

The present invention permits one to combine ESS measurements with techniques for tissue treatment or removal. For example, ESS measurements may be employed as a guide to identify optimum sites for tissue removal and/or determine that tissue removal is not necessary. Accordingly, the embodiments of the present invention facilitate the incorporation of ESS measurements with techniques for tissue treatment or removal. In particular, embodiments may integrate optical probes used for ESS measurements with a tissue manipulation apparatus, such as forceps, or a snare, a needle, etc., which operates on the examined tissue. Such embodiments enable ESS spectra to be collected and accurately co-registered to tissue treatment or diagnosis, such as biopsy. Preferably, the optical probes employed in these embodiments are multi-fiber optical probes adapted to make contact with the examined tissue to record ESS measurements. Preferably, one uses a disposable device containing the fibers, the tissue manipulation apparatus and a connector to the full equipment.

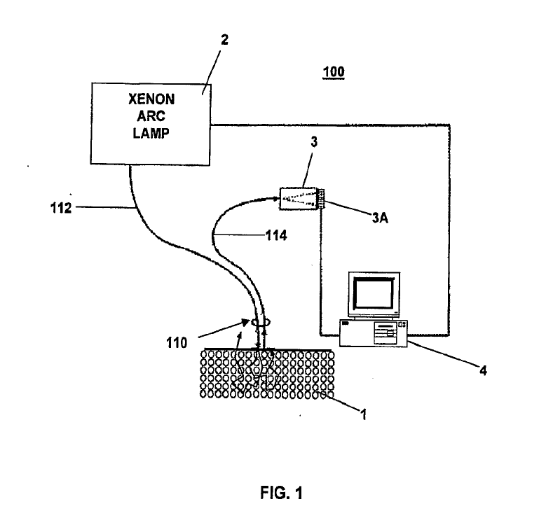

In general, ESS measurements may be performed via fiberoptic probes, which enable in vivo screening and identification of tissues. FIG. 1 illustrates an embodiment of an ESS diagnostic system. The ESS system 100 employs a fiberoptic probe 110, a pulsed xenon arc lamp 2, an analyzing spectrometer 3 including a linear charge-coupled device (CCD) array 3A for detection, and a computing/interface device 4, such as a personal computer or embedded computer. Generally, the fiberoptic probe 110 is employed to transmit light to, and collect light from, a target tissue site 1 to make ESS measurements. The pulsed xenon-arc lamp 2 provides an illumination source for the fiberoptic probe 110. The spectrometer 3 with detector 3A collects and analyzes the light collected by the fiberoptic probe 110. The computing/interface device 4 helps control the system and displays data. In some embodiments, the arc lamp 2, the spectrometer 3 with detector 3A, the computing/interface device 4, and a power supply for the system (not shown) may be conveniently housed in a briefcase sized unit for portability.

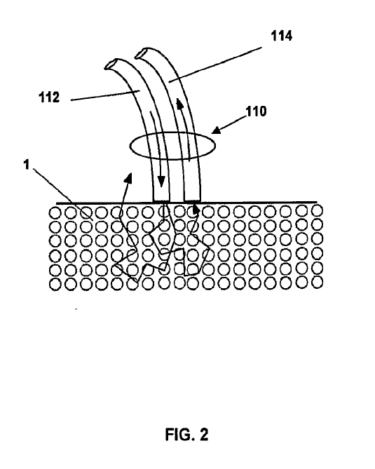

As shown in FIGS. 1 and 2, the fiberoptic probe 110 for ESS measurements may be designed to be in optical contact with the tissue 1 being examined. Alternatively, a standoff spacer may be employed to position the tip of the fiberoptic probe 110 a small distance from, rather than in direct contact with, the tissue. As further illustrated by FIGS. 1 and 2, the fiberoptic probe 110 may employ at least one flexible illuminating fiber 112 for transmitting source illumination from the arc lamp 2 to the tissue 1, and at least one flexible collection fiber 114 for collecting the photons from the source illumination that undergo scattering by the tissue 1. In this embodiment, the optical fibers 112 and 114 are selected for their ability to transmit broadband light over the spectral range required. Because the fiberoptic probe 110 has at least two fibers, it is referred to as a multi-fiber optical probe. The multi-fiber optical probe 110 examines only the site that it contacts and does not image the tissue surface. By placing the multi-fiber optical probe 110 in direct contact with the tissue 1, surface Fresnel reflections are avoided and all of the collected photons are the result of photons that undergo one or more scattering events while making their way from the illumination fiber 112 to the collection fiber 114 through the tissue.

Advantageously, with separate illumination and collection fibers 112 and 114, fiber feedback is minimized and system-response calibration becomes simple and robust. In general, system calibration and field use is easier with multi-fiber probes as compared to single fiber probes, especially when white-light scattering measurements are recorded.

In order to ensure both accurate and reproducible measurements within the clinical setting, the computing/interface device 4 may control the entire measurement process, which includes activating the spectrometer 3, triggering the arc lamp 2, and reading the detector array 3A with an analog/digital (A/D) converter. The computing/interface device 4 itself may be activated by an operator through a foot pedal or a key stroke. The computing/interface device 4 also permits rapid data acquisition, analysis, and graphical display.

As illustrated in FIG. 1, the ESS system 100 may direct short pulses, typically 1-30 μs in duration, of white light from the pulsed xenon arc lamp 2. In this embodiment the light from the xenon arc lamp is filtered and has a wavelength range of approximately 320 to 920 nm, but, in other embodiments, the ESS system 100 may use other wavelength ranges, depending on the tissue type and diagnostic application. In a particular embodiment, the wavelength range is approximately 320 to 850 nm. If desired for safety, ultraviolet B (280 to 315 nm) and ultraviolet C (100 to 280 nm) may be filtered out to avoid any potential risk to patients. The output light of the arc lamp 2 is filtered if necessary and coupled to the illumination fiber 112 of the multi-fiber optical probe 110, which transmits photons to the targeted tissue site 1. For many ESS applications the targeted tissue site 1 typically has a volume that is not greater than 0.05 mm³. The collection fiber 114 collects a small fraction of the scattered light from the tissue 1 and transmits the collected light to the analyzing spectrometer 3, which generates the optical spectrum for further processing by the computing/interface device 4.

While some embodiments described herein may employ broadband light, it is understood that other embodiments may transmit and detect light from an illumination source that provides light having discrete, or specific, wavelengths.

The collection and recording of a single spectrum typically takes less than a quarter of a second, and can be as short as 10 ms or shorter. The use of a pulsed short-arc lamp 2 permits a short integration time and reduces interference from background light. Typical data acquisition and display time by the computing/interface device 4 is less than 300 ms for each site measurement. It is possible to perform two or more measurements per second, limited by the time to move the multi-fiber optical probe from spot to spot.

In the current embodiment, before any spectra are recorded for the tissue being examined, a reference spectrum, e.g., a white spectrum, is established by recording the diffuse reflectance from a reference material, such as a surface of Spectralon™ (Labsphere, Inc.), which is spectrally flat at least between 250 and 1000 nm. The reference spectrum establishes the system response and is used to account for spectral variations in the light source, spectrometer, fiber transmission, and fiber coupling.

In addition, in this embodiment, less than 100 ms before any spectral measurement (reference or tissue) is recorded, the system records a “dark” spectrum with no illumination from the xenon arc lamp, which is subtracted from the subsequent spectral recording with illumination from the lamp. Therefore, in this embodiment, the tissue spectrum that is stored and displayed is determined by: (Sₜᵢₛₛᵤₑ−Dₜᵢₛₛᵤₑ)/(S_{reference}−D_{reference}), where _{reference} indicates a measurement with the reference material, tissue indicates a measurement of the tissue, S indicates a spectrum recording with illumination from the lamp, and D indicates a dark recording with no illumination from the lamp. This approach accounts for the site-specific ambient light at the time of measurement as well as the detector dark current.

The multi-fiber optical probe 110 employs a fixed center-to-center separation between the tips of the illuminating fiber 112 and the collection fiber 114 in contact with the tissue. With the appropriate optical geometry, ESS can report the size, structure, and index of refraction of subcellular components. Because the cellular components that cause elastic scattering have dimensions typically on the order of visible to near-infrared wavelengths, the elastic scattering properties will exhibit a wavelength dependence that is more complex than for simple (1/λ⁴) Rayleigh scattering. When the illumination and collection fibers are sufficiently separated for the diffusion approximation to be valid (typically ≧0.5 cm), the spectral dependence of the collected light will be less sensitive to the size and shapes of the scattering centers. However, for small separations (≦0.1 cm), the wavelength dependence is sensitive to tissue micromorphology. Accordingly, for such geometries, morphology and size changes can be expected to cause significant changes in an optical signature due to the wavelength dependence of elastic scattering. As Rayleigh approximations are not suitable for mathematical simulations in this case, the details of the scattering events are determined from computational simulations, such as Monte Carlo simulations, incorporating Mie theory. For example, in the clinical diagnosis of breast cancer, it has been determined that for a separation of 350 μm between the illumination and collection fibers, the volume of examined tissue visited by the collected photons occupies a zone approximately 500 μm long, 300 μm wide, and 300 μm deep. In general, the close proximity of the fibers (≦350 μm, center-to-center) is beneficial to providing sufficient sensitivity of ESS to sub-cellular structural characteristics.

With certain ESS probe geometries, the resulting effective path length of the collected photons is generally several times greater than the actual separation of the probe fibers in contact with the tissue. As a result, the system has good sensitivity to the optical absorption bands of the tissue components, adding valuable complexity to the scattering spectral signal.

Designs for ESS multi-fiber optical probes may specify the diameters of the illumination and collection fibers, in addition to the separation distance between centers of the fibers and angles of the fibers to each other and the tissue surface facets. Various embodiments of the multi-fiber optical probe assembly measure approximately <1 mm to 5 mm in diameter. In a particular embodiment, the core diameter of the illumination fiber is approximately 400 μm, while the core diameter of the collection fiber is approximately 200 μm. Furthermore, in this embodiment, the centers of the optical fibers may be separated by 350 μm, and the two fibers may be encased in a sheath to form a multi-fiber optical probe approximately 1-2 mm in diameter. Meanwhile, in another embodiment, the illumination fiber and the collection fiber each have a core diameter of approximately 200 μm and the multi-fiber optical probe is approximately less than 0.6 mm in diameter.

Geometric parameters, such as fiber separation, fiber diameter, fiber angle, and fiber facet angle, can be independently controlled to control of depth sensitivity and volume being measured. The geometric parameters of the multi-fiber optical probe design are significant factors in the characteristic spectra obtained for a particular tissue. As a result, these parameters are generally fixed at least for each clinical study in order to optimize the sensitivity of the system to changes in the tissue and to prevent variations between spectral recordings that result from instrumental artifacts.

Applying ESS is not limited to employing multi-fiber probes that use one illumination fiber and one collection fiber. The use of two collection fibers enables the use of polarization subtraction for separation of a single-scattering signal from a multiple-scattering signal, which in turn permits better isolation of the epithelial signal from deeper tissues. Moreover, a fiber as described herein may actually refer to a bundle of individual fibers, all of which work together to illuminate the tissue or collect the scattered photons.

Two different multi-fiber probe configurations have been used for clinical implementation and experimental studies. The difference between the two designs lies in the mechanical housing of the multi-fiber probe, which allows them to be optimized for different clinical applications. The first design incorporates a larger stainless-steel sheath, about 5 mm in diameter, to house the optical fibers. This ergonomically convenient, hand-held, pen-like design enables measurements during open surgery. A second design, however, is required for interstitial (transdermal) measurements. These multi-fiber probes have a small-diameter (≦1 mm) stainless-steel outer sheath, which houses the optical fibers. The outer diameter of these multi-fiber probes is carefully chosen to be compatible with the inner bore of current core-biopsy needles conventionally used in clinics, in order to permit the multi-fiber probe to be passed easily and safely down the needle and presented to the tissue under examination.

Multi-fiber probes of the small-diameter design are passed endoscopically to the tissue of interest and ESS measurements are taken. When biopsies are also required using the prior art procedure, the multi-fiber probe is then withdrawn and forceps are passed through the endoscopic device to obtain a pinch biopsy of what is a best estimate of the spot where the ESS measurements were taken. In other words, this procedure requires the multi-fiber probe to be withdrawn for the forceps each time tissue is removed. As noted previously, however, ESS is a site-specific measurement—not an imaging modality—that samples a tissue volume typically of ≦0.05 mm³. However, when this particular procedure is applied, with separate fiber probe and forceps tools, the tissue removed by the forceps does not always coincide with the ESS measurement spot with the desired accuracy. In addition, additional trauma to the subject can be inflicted by this procedure. Embodiments of the present invention avoid such problems.

Moreover, surveillance of larger areas with ESS requires making many point measurements in rapid succession. However, sweeping measurements requiring the removal of tissue samples is slowed considerably by the need to withdraw the multi-fiber probe for the forceps after each measurement.

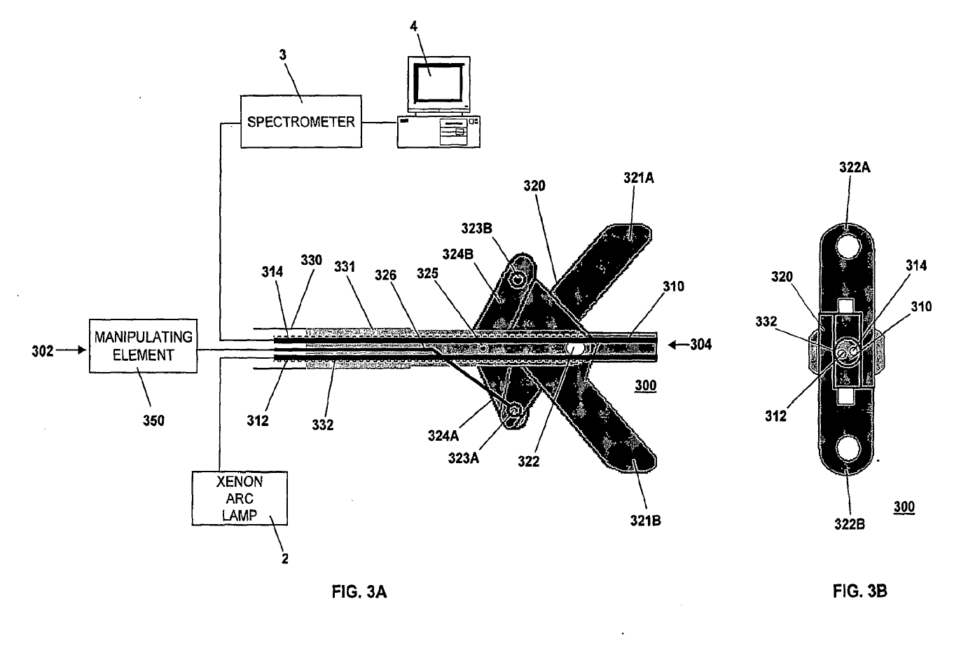

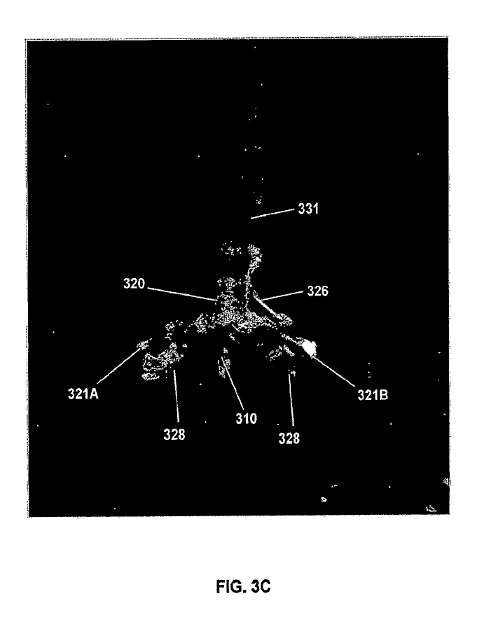

Accordingly, to address the disadvantages of having to withdraw the multi-fiber probe for the forceps or manipulation device after each measurement, embodiments of the present invention enable ESS spectra to be collected and accurately co-registered to tissue treatment or diagnosis, such as biopsy. One such embodiment is schematically illustrated by FIGS. 3A and 3B. A picture of a similar embodiment is also shown in FIG. 3C. As FIGS. 3A and 3B shows, an optical forceps device 300 integrates an ESS multi-fiber optical probe 310, as described previously, with a surgical biopsy forceps 320. Advantageously, the optical forceps device 300 greatly increases the accuracy of tissue removal from the precise spot from which ESS measurements are recorded and shortens the time required to remove tissue from the ESS measurement spot with the forceps 320.

As illustrated in FIG. 3A, the optical forceps device 300 may be employed as an endoscope-mediated device. As such, the optical forceps device 300 may be conveyed through a flexible sheath 330 which extends longitudinally from a proximal end 302 to a distal end 304. The sheath 330 may be formed with Teflon. In addition, a hypotube 331, which may be formed of surgical metal, such as stainless steel, may be employed at the distal end 304. In general, the distal end 304 is the leading end that is introduced into the body and engages the tissue under examination.

The biopsy forceps 320 of the optical forceps device 300 is positioned at the distal end 304 and includes jaws 321A and 321B which extend from the hypotube 331. The sheath 330 and the hypotube 331 enable the biopsy forceps 320 to be guided into position over a tissue sample and to execute a biopsy of the sample tissue. The jaws 321A and 321B are operably attached to the hypotube 331 via a front hinge 322. The jaws 321A and 321B pivot about the front hinge 322 in opposing directions to move between an open position where the jaws 321A and 321B are spaced apart, as shown in FIG. 3A, and a closed position where the jaws 321A and 321B come together into contact, or near contact. The movement of jaws 321A and 321B enables the forceps 320 to act on a target tissue sample positioned at the distal end 304. Where required, sufficient pressure may then be applied to detach the tissue from the corresponding body part for biopsy.

When they are in the closed position, the jaws 321A and 321B may be substantially aligned with the hypotube 331 in the longitudinal direction. In addition, at least one of the jaws 321A and 321B may have a structure, such as cutting teeth, on a surface facing the other jaw, so that the structure is able to execute a biopsy on the target tissue when the jaws 321A and 321B are moved into the closed position. An example of such a structure is illustrated by reference numeral 328 in FIG. 3C.

In the example of FIGS. 3A and 3B, the jaws 321A and 321B also extend from the front hinge 322 to side hinges 323A and 323B where they are coupled to rear arms 324A and 324B, respectively. At ends opposite to the sides hinges 323A and 323B, the rear arms 324A and 324B may be coupled together at a rear hinge 325. A control wire 326 extends from a manipulating element 350 to the forceps 320 to operate the rear arms 324A and 324B. As is known with conventional endoscopic devices, the manipulating element 350 may include a scissor-like handle, pistol grip, or the like, for operating a wire or other actuator to cause the movement of the jaws 321A and 321B.

Typically, the control wire 326 extends longitudinally within a central channel 332 of the sheath 330 and the hypotube 331. As shown in FIG. 3A, the control wire 326 exits from the side of the hypotube 331 to be coupled to the side hinge 323A. As such, the control wire 326 may be operated to move both the jaw 321A and the rear arm 324A which are both coupled at side hinge 323A. With movement of rear arm 324A, the rear arm 324B and thus jaw 321B also move. As a result, the control wire 326 may be employed to move both jaws 321A and 321B. Indeed, as shown in FIG. 3A, the jaws 321A and 321B and the rear arms 324A and 324B are all directly or indirectly coupled in a manner so that any movement of one of the elements causes corresponding movement of the other elements. Therefore, in alternative embodiments, one or more control wires 326 may be coupled to any one of the jaws 321A and 321B and the rear arms 324A and 324B to operate the forceps 320.

An operator works the manipulating element 350 at the proximal end 302 to cause the control wire 326 to move longitudinally in either direction with respect to the hypotube 331. When the control wire 326 moves longitudinally toward the distal end 304, the side hinge 323A correspondingly moves toward the distal end 304 and curves away from the hypotube 331, causing the jaw 321A to pivot about the front hinge 322 toward the open position. Meanwhile, the rear arm 324A moves to cause the rear hinge 325 to move longitudinally toward the distal end 304. This movement of the rear hinge 325 causes the rear arm 324B to push the side hinge 323B. The side hinge 323B then also moves toward the distal end 304 and curves away from the hypotube 331. With the movement of the side hinge 323B, the jaw 321B pivots about the front hinge 322 toward the open position. Accordingly, movement of the control wire toward the distal end 304 causes the jaws 321A and 321B to move into the open position.

On the other hand, when the control wire 326 moves longitudinally away from the distal end 304, the side hinge 323A correspondingly moves away from the distal end 304 and curves toward the hypotube 331, causing the jaw 321A to pivot about the front hinge 322 toward the closed position. Meanwhile, the rear arm 324A moves to cause the rear hinge 325 to move longitudinally away from the distal end 304. This movement of the rear hinge 325 causes the rear arm 324B to pull the side hinge 323B. The side hinge 323B then also moves away from the distal end 304 and curves toward the hypotube 331. With the movement of the side hinge 323B, the jaw 321B pivots about the front hinge 322 toward the closed position. Accordingly, movement of the control wire away from the distal end 304 causes the jaws 321A and 321B to move into the closed position.

In an alternative embodiment, the optical forceps device 300 may employ a forceps 320 that does not have the rear arms 324A and 324B. Rather, two control wires 326 are coupled to each of the jaws 321A and 321B, and movement of the jaws 321A and 321B is caused by operation of both control wires 326.

As discussed previously, the optical forceps device 300 is employed with a central channel 332 extending through the sheath 330 and the hypotube 331. In addition to providing a passageway for the control wire 326, the central channel 332 also enables a multi-fiber optical probe 310 to be conveyed to the distal end 304 of the optical forceps device 300. Therefore, in the manner described previously, the tip of the multi-fiber optical probe 310 is positioned to take ESS measurements of a tissue sample at the distal end 304. In particular, the multi-fiber optical probe 310 includes at least one illumination fiber 312 and at least one collection fiber 314 which extend into an area between the jaws 321A and 321B, as shown in FIGS. 3A and 3B. The multi-fiber optical probe 310 does not interfere with the function of the jaws 321A and 321B, as the jaws 321A and 321B are shaped to maintain a space for the multi-fiber optical probe 310 even when they move into the closed position. As discussed above, additional structures may be positioned on the jaws 321A and 321B to enable the forceps 320 to engage a tissue sample while other parts of the jaws 321A and 321B remain spaced apart for the multi-fiber optical probe 310.

As further illustrated in FIG. 3A, the illumination fiber 312 and the collection fiber 314 extend from the forceps 320 toward the proximal end 302 and are coupled to a xenon arc lamp 2 and to a spectrometer 3, respectively. Thus, as also discussed previously, the xenon arc lamp 2 may direct short pulses, approximately 1-30 μs in duration, of white light. The output light from the arc lamp 2, which may have a wavelength range of approximately 320 to 920 nm, is coupled to the illumination fiber 312 of the multi-fiber optical probe 310. The illumination fiber 312 transmits photons at the distal end 304 where the targeted tissue site is positioned. The collection fiber 314 collects a small fraction of the scattered light from the tissue and transmits the collected light to the analyzing spectrometer 3 and detector array 3A, which generates the optical spectrum for further processing by the connected computing/interface device 4.

The specifications for the core diameter of the illuminating or collection fiber and for the separation between the fibers are optimized for the tissue being examined. An optical forceps device 300 may be manufactured with a particular set of geometric parameters suited for a particular application. Moreover, the optical forceps device 300 can be small enough to sample tissue from small animals, such as rodents that are used in validation studies.

Depending on the desired application, the tip of the multi-fiber optical probe 310 can be moved longitudinally within the central channel 332 or fixed to protrude between the jaws 321A and 321B. After identifying a region of interest, one of two approaches may be taken. If the multi-fiber optical probe 310 is moveable, the open jaws 321A and 321B are placed in apposition to the tissue being examined and the multi-fiber optical probe 310 is advanced through the central channel 332 to make contact with the tissue. After measurements are obtained, the jaws 321A and 321B are moved from the open position to a closed position, and the tissue is avulsed to provide the tissue sample for biopsy. Alternatively, another type of tissue manipulation, such as cautery ablation, can be implemented in place of avulsion by the jaws 321A and 321B. For instance, the device 300 can employ a radio-frequency (RF) energy mechanism to perform ablation or electrocautery of tissue.

If, however, the multi-fiber optical probe 310 is fixed as in a multi-bite “spike” located between the jaws 321A and 321B, the area of interest is grasped first between the jaws 321A and 321B while ESS measurements are obtained. Based on the ESS measurement obtained, the grasped tissue may be released, avulsed for tissue removal, and/or ablated. This fixed probe technique may produce more consistent, reproducible measurements as the quantity and orientation of the tissue within the closed jaws 321A and 321B is not subject to appreciable variation.

One alternative embodiment of the optical forceps 300 may be equipped with an extendible needle which has a microscopic mirror which can be used to alter or control the transmission of light by the fibers of the multi-fiber probe. The microscopic mirror may be used to redirect the illumination light, for instance by 90 degrees, to achieve a different geometric orientation. In addition, the mirror may be used to increase the area of illumination. If, the diameter of the fibers is extremely narrow, for example 100 μm, the mirror can be used to make the effective illumination greater than 100 μm.

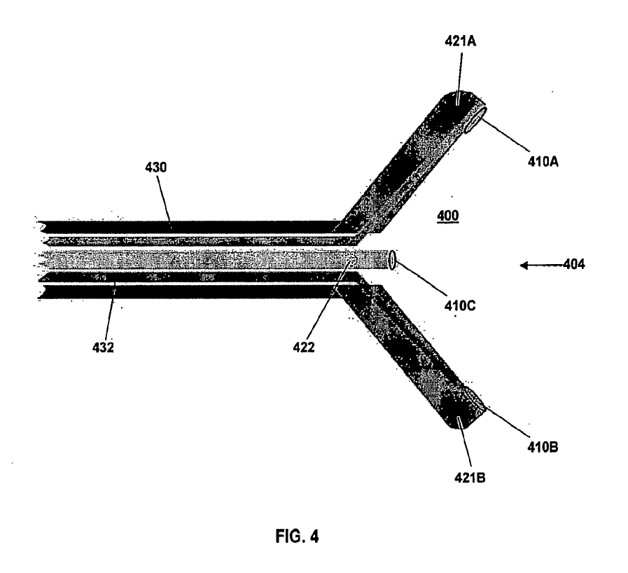

In yet another embodiment of the present invention, a multi-probe optical forceps device 400, as illustrated in FIG. 4, employs a plurality of multi-fiber optical probes 410A, 410B, and 410C with a single forceps 420. As with the optical forceps device 300 shown in FIG. 3, the device 400 may be an endoscopic device which includes a jaw-type biopsy forceps 420 with jaws 421A and 421B at a distal end 404. Unless indicated otherwise, the multi-probe optical forceps device 400 is similar to the optical forceps device 300 described previously.

A central channel 432 passing through a sheath 430 enables a plurality of multi-fiber optical probes 410A, 410B, and 410C to extend to the distal end 404 and make ESS tissue measurements from multiple areas about the distal end 404. Each of the multi-fiber optical probes 410A, 410B, and 410C has at least one illumination fiber and at least one collection fiber to make ESS measurements in the manner described previously. Like the optical forceps 300 shown in FIG. 3, a multi-fiber optical probe 410C is positioned between the jaws 421A and 421B near the front hinge 422 and can operate like the multi-fiber optical probe 310 in optical forceps 300. However, with the multi-probe optical forceps device 400, additional multi-fiber optical probes 410A and 410B are provided respectively within the jaws 421A and 421B, with the tip of each multi-fiber probe fixed at the end of the jaws 421A and 421B. The additional multi-fiber probes 410A and 410B allow ESS measurements to be conveniently taken from more than one tissue site simultaneously or near simultaneously. Moreover, the additional multi-fiber probes 410A and 410B permit spectral data to be recorded with a variety of optical orientations, especially for backscattering and forward-scattering measurements. Although a plurality of multi-fiber probes 410A, 410B, and 410C are employed with the device 400 and the central channel 432 may be larger than those for the optical forceps device 300, the size of the device 400 remains sufficiently small to make its use feasible.

The multi-probe optical forceps device 400 is particularly advantageous as a research tool, because it provides a plurality of optical geometries for tissue scattering studies. With the jaws 421A and 421B open or closed in varying degrees in contact with the tissue, backscatter and forward scatter measurements may be obtained depending on the array of exciting and collecting fibers selected. These measurements, separately or in combination, can then be correlated with histopathological changes in the tissue.

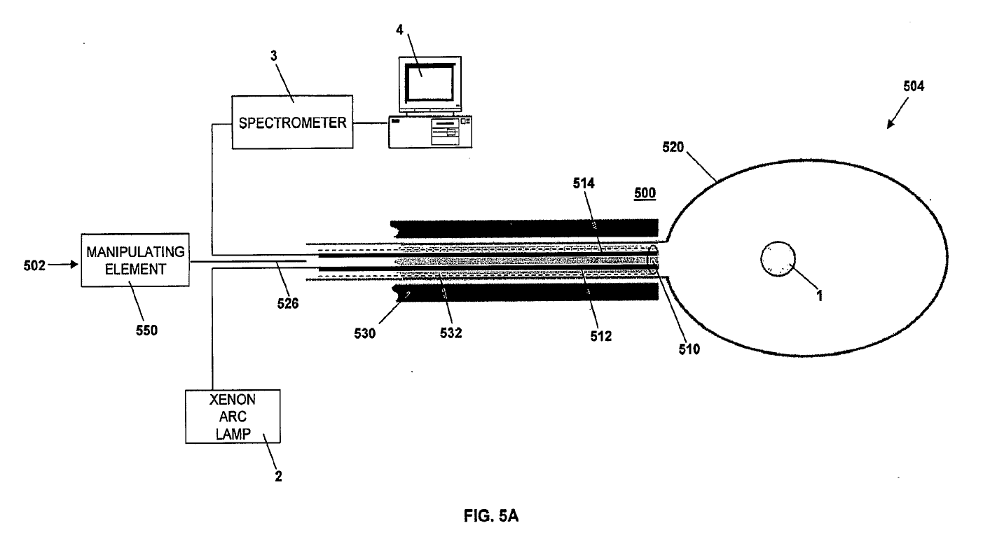

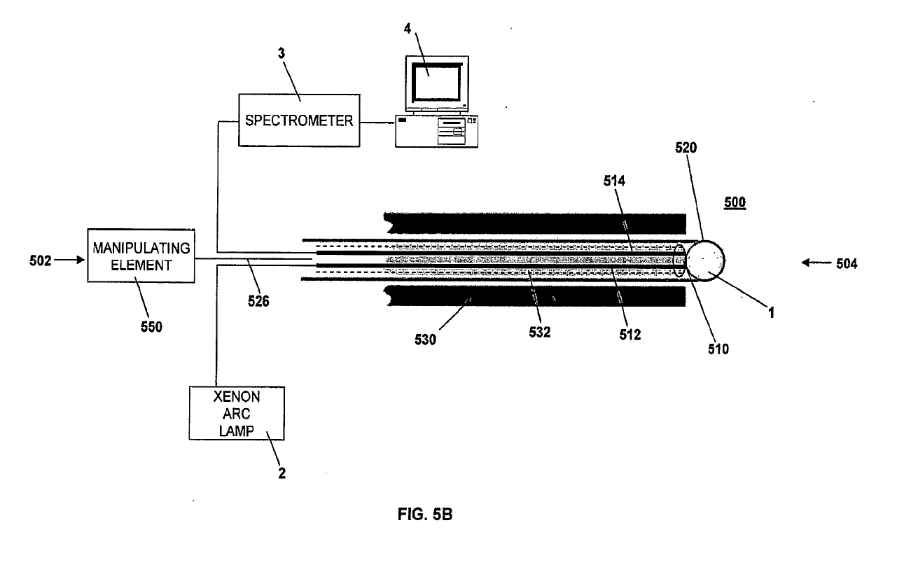

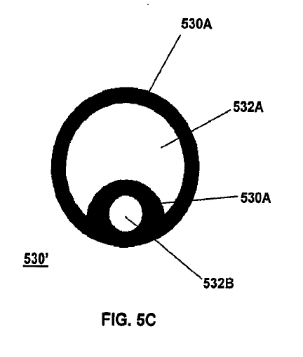

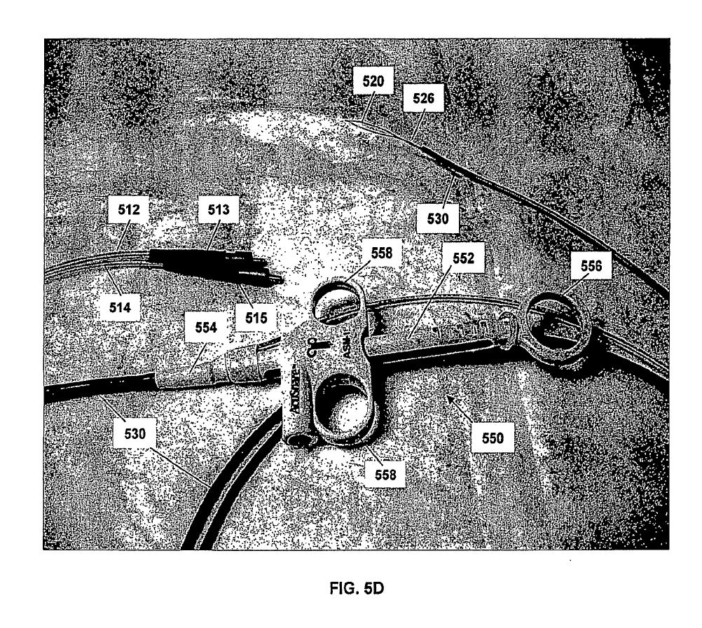

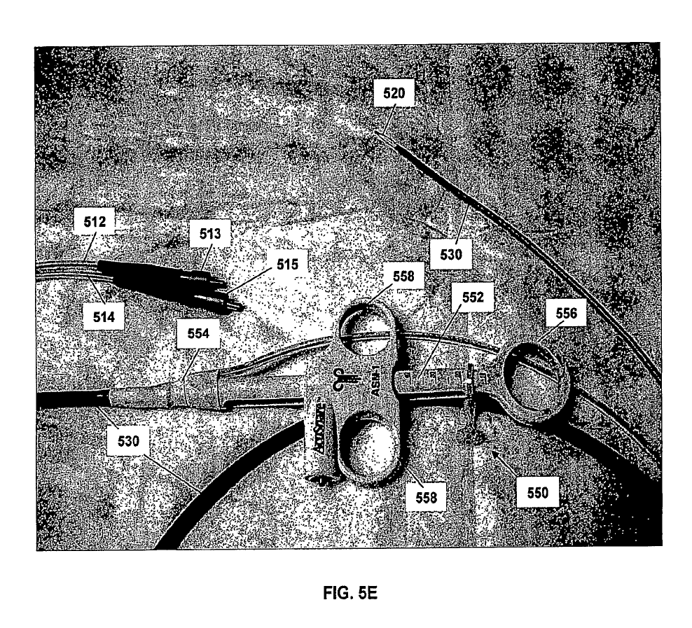

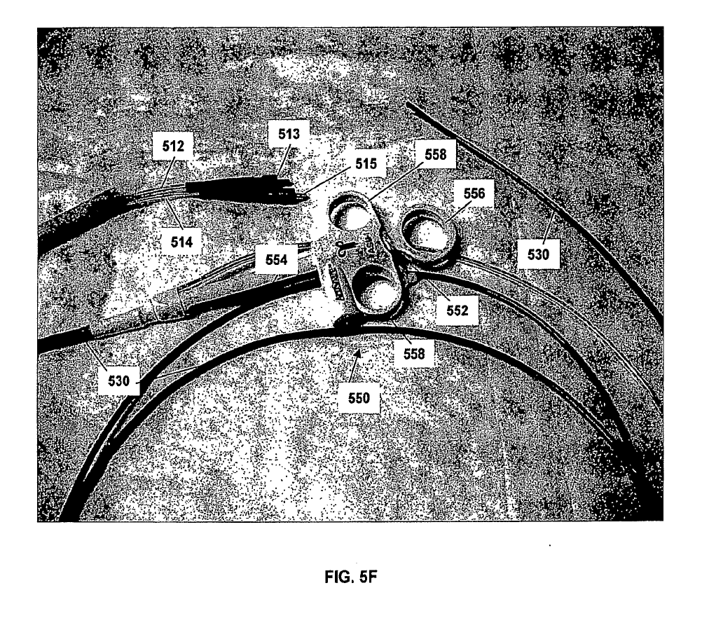

As indicated previously, the present invention integrates a multi-fiber probe with a variety of tissue manipulation methods. Thus, rather than employing forceps, an embodiment of the present invention, as illustrated in FIGS. 5A-F, may be an endoscope-mediated device that includes a polypectomy-type snare catheter 520 with a multi-fiber probe 510. The snare device 520 may be a nickel-titanium “shape-memory alloy” wire loop. For example, the snare device 520 of the optical snare device 500 may be used to remove tissue, such as pedunculated polyps 1, identified during colonoscopy.

Referring to FIGS. 5A and 5B, the optical snare device 500 may be an endoscope-mediated device. As such, the optical forceps device 500 may be conveyed through a flexible sheath 530 which extends longitudinally from a proximal end 502 to a distal end 504. The sheath 530 may be formed with Teflon. In general, the distal end 504 acts as the leading tip that is introduced into contact with the tissue 1 under examination. Therefore, the snare device 520 is positioned at the distal end 504. On the opposite proximal end 502, a manipulating element 550 is positioned to enable an operator to actuate and move the snare device 520 between the two positions shown in FIGS. 5A and 5B. In FIG. 5A, the snare device 520 is disengaged from the tissue, or polyp 1. On the other hand, in FIG. 5B, the snare device 520 engages the polyp 1.

As is known with conventional endoscopic devices, the manipulating element 550 may include a scissor-like handle, pistol grip, or the like for actuating the snare device 520. Typically, to operate the snare device 520, a control wire 526 extends within a central channel 532 from the manipulating element 550 in the sheath 530 to the snare device 520. An example of a manipulating element 550 is illustrated in FIGS. 5D-F. As shown in FIGS. 5D-F, the manipulating element 550 employs a plunger element 554 that moves relative to a connecting body 552 which is operably connected to the sheath 530. The plunger element 554 is operably connected via a wire 526 to the snare device 520 positioned at the distal end 504. When the plunger element 554 moves relative to the connecting body 552, the snare device 520 moves relative to the sheath 530. As the snare device 520 moves relative to the sheath 530, the loop that makes up the snare device 520 moves in and out of the sheath 530, causing the size of the loop to move between the sizes illustrated in FIGS. 5A and 5B. An operator may place a thumb in the thumb hole 556 and two fingers in the two finger holes 558. Because the thumb hole 556 is attached to the plunger element 554 and the finger holes 558 are attached to the connecting body 552, the operator may cause relative movement between the plunger element 554 and the connecting body 552 through relative movement between the thumb and two fingers. FIG. 5D illustrates the manipulating element 550 fully deploying the snare device 520 from the sheath 530. FIG. 5E illustrates the manipulating element 550 partially retracting the snare device 520 from the sheath 530. FIG. 5F illustrates the manipulating element 550 fully retracting the snare device 520 from the sheath 530.

As discussed previously, the optical snare device 500 is employed with a central channel 532 extending through the sheath 530. In addition to providing a passageway for the control wire 526, the central channel 532 also enables a multi-fiber optical probe 510 to be passed to the distal end 504 of the optical snare device 500. Therefore, in the manner described previously, the tip of the multi-fiber optical probe 510 is positioned to take ESS measurements of a tissue sample at the distal end 504. In particular, the multi-fiber optical probe 510 includes at least one illumination fiber 512 and at least one collection fiber 514, as shown in FIGS. 5A and 5B.

As further illustrated in FIGS. 5A and 5B, the illumination fiber 512 and the collection fiber 514 extend from the distal end 504 toward the proximal end 502 and are coupled to a xenon arc lamp 2 and to a spectrometer 3, respectively. Thus, as also discussed previously, the xenon arc lamp 2 may direct short pulses, approximately 1-30 μs in duration, of white light. The output light from the arc lamp 2, which may have a wavelength range of approximately 320 to 920 nm, is coupled to the illumination fiber 512 of the multi-fiber optical probe 510. The illumination fiber 512 transmits photons to the distal end 504 where the targeted tissue site is positioned. The collection fiber 514 collects a small fraction of the scattered light from the tissue and transmits the collected light to the analyzing spectrometer 3 and detector array 3A, which generates the optical spectrum for further processing by the connected computing/interface device 4.

In a particular embodiment, the optical snare device 500 may employ a dual-lumen tubing 530′ to guide the control wire 526 for the snare device 520 and the multi-fiber optical probe 510 from the proximal end 502 to the distal end 504. As illustrated by the cross-section of FIG. 5C, two lumens, or channels, 532A and 532B are defined by outer wall 530A and inner wall 530B. Thus, the control wire 526 and the multi-fiber optical probe 510 are situated in separate passageways. In a particular embodiment, the illuminating fiber and the collection fiber are each a 200 μm optical fiber. Thus, the control wire 526 may pass through the larger channel 532A while the multi-fiber optical probe 510 may pass through the smaller channel 532B. The dual-lumen tubing 530′ may be formed from a polyethylene blend (PEBAX) through an extrusion process.

Typically, the snare device 520 is lassoed over the top of the polyp and pulled snugly around the stalk. The stalk is then “garroted” by pulling the wire until the stalk is transected, with or without electrocautery. Without the optical snare device 500, the tissue must be retrieved and forwarded to pathology, fixed, sectioned, stained, and assessed for dysplastic or neoplastic tissue within the polyp.

The optical snare device 500, as shown in FIGS. 5A and 5B, identifies the lower border of dysplastic tissue prior to transection of the stalk. As shown particularly in FIG. 5B, both sessile and pedunculated polyps are lassoed and the tissue intended for transaction is pulled in contact with the multi-fiber probe 510 for pre-polypectomy ESS measurements. The optical snare device 500 can be used for the piecemeal removal of flat and sessile polyps as well, where mucosa bunched up and grasped by the snare is assessed for dysplastic mucosa and removed. Subsequent resections of the surrounding tissue are guided by ESS measurements to ensure that all dysplastic tissue has been excised and/or ablated.

Further embodiments of the present invention may include disposable and replaceable components to minimize cross-contamination and to facilitate preparation for procedures. In general, the arc lamp 2, the spectrometer 3, the computing/interface device 4, and similar components may be used repeatedly, especially when considering their cost. Indeed, as discussed previously, many of these components can be conveniently arranged in a briefcase sized package which facilitates transport and reuse. On the other hand, it may be preferable to discard other components after a single use on one patient. In particular, components that are likely to come into contact with the patient's tissue and bodily fluids are preferably discarded to minimize cross-contamination. For example, as shown in FIGS. 5D-F, a tissue manipulation apparatus, such as an optical snare device 500 employs SMA connectors, or couplers, 513 and 515 to detachably connect the illuminating fiber 512 and the collection fiber 514 to the arc lamp 2 and the spectrometer 3, respectively. The connectors 513 and 515 enable the assembly shown in FIGS. 5D-F to be easily removed from to the arc lamp 2 and the spectrometer 3 and discarded. Furthermore, replacing a used assembly only requires simple connection of the connectors 513 and 515 to the arc lamp 2 and the spectrometer 3, respectively. In other words, the manipulating element 550, the sheath 530, the illuminating fiber 512 and the collection fiber 514, the control wire 526, and the snare 520 may be considered disposable. These components may also be relatively inexpensive to manufacture, from primarily plastic materials for example, thus making it more feasible to dispose of them after only one use.

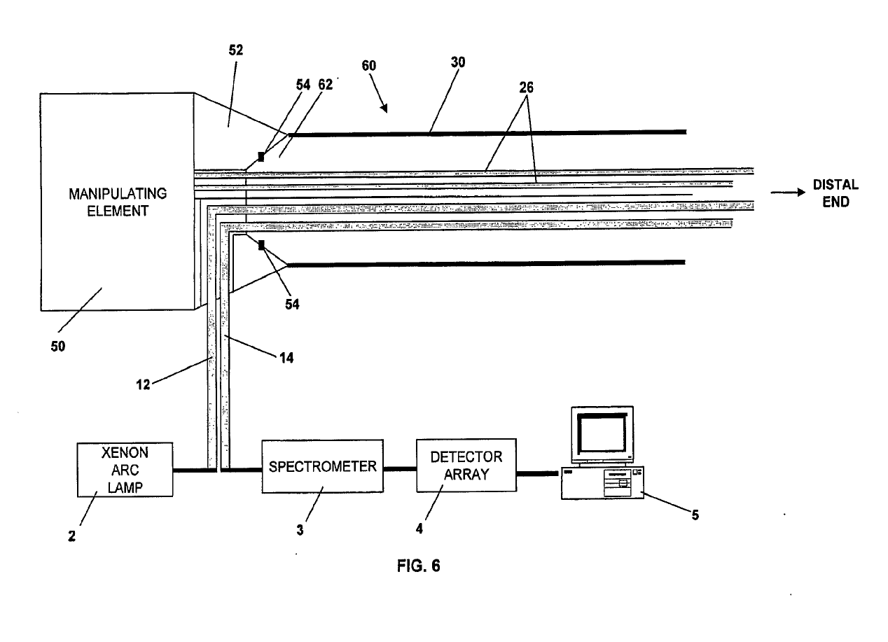

However, configurations of other embodiments provide other types of disposable assemblies. In general, a connection point, or interface, between disposable and reusable components may occupy different positions. For instance, FIG. 6 illustrates an embodiment where the manipulating element 50 is not disposable and may be reused. Here, the disposable assembly 60 includes components such as the sheath 30, a portion of the illuminating fiber 12, a portion of the collection fiber 14, the control wire (not shown), and the tissue manipulation apparatus, such as a forceps or snare (not shown). As shown in FIG. 6, the disposable assembly 60 is detachably connected to the manipulating element 50 so that the manipulating element 50 may be reused along with the arc lamp 2, the spectrometer 3, and the computing/interface device 4. Thus, an interface between the disposable assembly 60 and the reusable components occurs at the manipulating element 50. In particular, the manipulating element 50 has a connecting body 52 that receives a corresponding connecting end 62 of the disposable assembly 60. The connecting end 62 and the connecting body 52 may have corresponding shapes for engaging each other. Once the connecting end 62 and the connecting body 52 are engaged, locks 54 ensure that the connecting end 62 and connecting body 52 are detachably connected but that the parts will not suffer axial separation under normal operation of the ESS system and the tissue manipulation apparatus. The locks 54 may employ, without limitation, a treaded engagement, frictional engagement, locking pins, locking tabs, fasteners, adhesives, mechanically interlocking parts, or the like. Although additional positioning guides, interlocking parts, or other structures may also be employed, the locks 54 may also prevent relative coaxial rotation between the connecting body 52 and the connecting end 62. In general, an operator must actively and purposely release the connecting end 62 and the connecting body 52 from engagement with each other.

As discussed above, wires or other actuators may be employed to enable the manipulating element 50 to operate the tissue manipulation apparatus at the distal end of the device. Therefore, as shown in FIG. 6, the wires 26 extend between the disposable assembly 60 and the manipulating element 50. The wires 26 are a part of the disposable assembly 60, but have a length that enables them to be connected to the manipulating element 50 but detached for later disposal.

As also shown in FIG. 6, the illuminating fiber 12 extends between the disposable assembly 60 and the arc lamp 2. Similarly, the collection fiber 14 extends between the disposable assembly 60 and the spectrometer 3. Like the embodiment of FIGS. 5D-F, the illuminating fiber 12 and the collection fiber 14 here are detachably connected to the arc lamp 2 and spectrometer 3, allowing them to be disconnected from the reusable components and to be disposable.

As described above, the computer/interface 4 may be a conventional personal computer or may be a dedicated computer embedded in the system and contained within the same housing as the other permanent components. In general, the computer/interface 4 may be a programmable processing device that executes software, or stored instructions, and that may be operably connected to the other devices, components, and sub-systems in accordance with the embodiments described above. Physical processors and/or machines employed by embodiments of the present invention for any processing or evaluation may include one or more networked or non-networked general purpose computer systems, microprocessors, field programmable gate arrays (FPGA's), digital signal processors (DSP's), micro-controllers, and the like, programmed according to the teachings of the exemplary embodiments of the present invention, as is appreciated by those skilled in the computer and software arts. The physical processors and/or machines may be externally networked with the image capture device, or may be integrated to reside within the image capture device. Appropriate software can be readily prepared by programmers of ordinary skill based on the teachings of the exemplary embodiments, as is appreciated by those skilled in the software art. In addition, the devices and subsystems of the exemplary embodiments can be implemented by the preparation of application-specific integrated circuits or by interconnecting an appropriate network of conventional component circuits, as is appreciated by those skilled in the electrical art(s). Thus, the exemplary embodiments are not limited to any specific combination of hardware circuitry and/or software.

Stored on any one or on a combination of computer readable media, the exemplary embodiments of the present invention may include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, and the like. Such computer readable media further can include the computer program product of an embodiment of the present inventions for performing all or a portion (if processing is distributed) of the processing performed in implementing the inventions. Computer code devices of the exemplary embodiments of the present inventions can include any suitable interpretable or executable code mechanism, including but not limited to scripts, interpretable programs, dynamic link libraries (DLLs), Java classes and applets, complete executable programs, and the like. Moreover, parts of the processing of the exemplary embodiments of the present inventions can be distributed for better performance, reliability, cost, and the like.

Common forms of computer-readable media may include, for example, a floppy disk, a flexible disk, hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read.

While the embodiments discussed herein have described embodiments of the present invention specifically in terms of elastic scattering spectroscopy, it is applicable to any spectral measurement technique that generally employs an illumination fiber to transmit photons into the examined tissue and a collection fiber to collect a sample of the photons after the photons have interacted with structures of the tissue. In addition, while the embodiments herein in particular may discuss removal of tissue, according to conventional biopsy, the present invention may employ other techniques for treating or manipulating the tissue, which may not require removal of the tissue. In general, the multi-fiber probe permits diagnosis of a tissue site before the technique is applied.

Methods for diagnosing and/or simultaneously treating tissues in a mammal, preferably a human utilizing the devices of the present invention are encompassed herein. In a preferred embodiment, the tissue is a tissue of the gastrointestinal (GI) tract or genitor-urinary (GU) tract. However, the tissue is not limited to these tissues and may include other organs, tissues, or cells.

For example, embodiments of the optical forceps device 300 are useful for endoscopic applications, such as procedures for esophageal or gastrointestinal treatment and diagnosis. As discussed, real-time ESS measurements enabled by embodiments of the present invention are clinically useful for increasing the pre-biopsy probability of obtaining the desired tissue, e.g., neoplastic tissue versus normal tissue. The optical forceps device 300 is advantageous for dysplasia surveillance strategies that require large numbers of random biopsies, such as procedures that are directed toward diagnosis and treatment of Barrett's esophagus or colonic dysplasia in inflammatory bowel disease. The optical forceps device 300 guides and refines the tissue sampling process, increasing detection yield and decreasing the total number of biopsies required for a given screening session. As such, the optical forceps device 300 also decreases the morbidity and overall cost of dysplasia surveillance. Similarly, the optical forceps device 300 assists in the assessment of diminutive colorectal polyps, permitting discrimination between hyperplastic versus adenomatous polyps prior to biopsy or ablation.

Colon cancer is the third most prevalent and deadly cancer in the U.S. (145,000 new cases and 56,000 deaths in 2005). Colon cancer develops from adenomatous polyps which grow into the lumen of the colon. Currently, endoscopists are unable to classify polyps based on appearance during routine video colonoscopy so all polyps are physically removed and collected for histopathology. Further screening or surveillance for colon cancer is based on a pathologist's subjective report. This process requires time, skill, risk, as well as considerable expense. While current practice is to remove all detected polyps, it is only the removal of adenomatous polyps that improves outcome. ESS can permit instantaneous identification of polyps such that not every polyp found would need to be removed.

In one study involving an embodiment of the optical forceps device 300, patients were enrolled from an extant pool referred for lower GI endoscopy. The optical forceps device 300 was used whenever endoscopic tissue sampling was indicated according to current standards of care. The optical forceps device 300 was employed to measure tissue according to ESS and to biopsy the measured tissue. The tissue was then submitted for standard histopathological diagnosis as well as for an independent secondary review to confirm histology. The optical biopsies were then correlated to the physical biopsies. The spectra obtained were classified with support vector machines (SVMs) using features extracted by performing principle component analysis (PCAs). The results for a total of 21 biopsies of colonic polyps (13 adenoma and 8 hyperplastic) were analyzed. Signal processing yielded a sensitivity of 84.62% and specificity of 75% for adenomatous vs. hyperplastic polyps. This study indicates that the optical forceps device 300 is able to co-register optical and physical biopsies. The study also indicates that the optical forceps device 300 is able to accurately and reliably differentiate dysplastic from non-dysplastic polyps of the colorectum. Accordingly, use of the device may help endoscopists target biopsies thus increasing the yield of tissue biopsy. If applied to current colon cancer screening recommendations, the device and system may decrease risks and costs of biopsy as well as save procedure time.

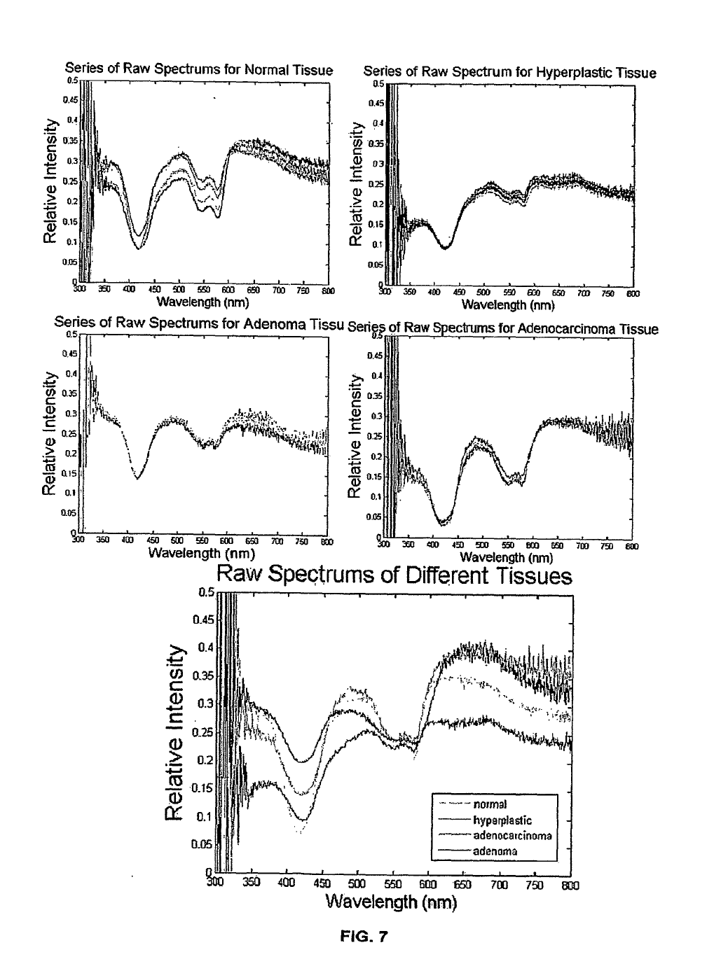

Another similar study employed the embodiment of the optical snare device 500, shown in FIGS. 5D-F. FIG. 7 illustrates the spectra representing plots of the relative intensity of scattered light vs. wavelength. The spectra are unsmoothed and normalized to the mean relative intensity. Different relative peak amplitudes are indicated for spectra corresponding to normal, hyperplastic, dysplastic (adenomatous) and, cancerous (adenocarcinoma) tissue. Thus, distinctive spectra have been obtained in vivo from colonic tissue using the optical snare device 500. The study demonstrates the feasibility of integrating small ESS multi-fiber optical probes, e.g. having two 200 μm fibers, into endoscopic snare devices. Integrated devices permit tissue classification prior to polypectomy/mucosal resection, thereby enhancing the efficiency of detecting dysplasia and colorectal cancer.

In another area, bladder cancer is the fourth highest occurring cancer in men and the seventh highest occurring cancer in women. It is found primarily in the elderly population and in smokers and over 60,000 new cases and 13,000 deaths per year. Bladder cancer has the highest recurrence rate of any cancer and the average total cost per patient is between $100,00 and $200,000, from diagnosis to death with a total cost of over $3.7 billion a year (Botteman et al., Pharmacoeconomics, 2003, 21(18):1315-30). Diagnosis is typically attempted for bladder cancer once a patient manifests hematuria. Hematuria generally yields a 10% cancer diagnosis. A step in the diagnosis of bladder cancer is obtaining a tissue biopsy. As such, almost 3 million cystoscopies are performed a year.

When bladder cancer is endoscopically resected, the margins of the lesion within the bladder are fulgerated and there is no definitive way to be certain that the tumor is entirely destroyed. Also, the depth of the resection must sometimes unnecessarily include muscle tissue surrounding the bladder if the physician is not sure of the depth of the cancer. This can be crucial for staging.

A device that gives real-time feedback about cancers, such as bladder cancer, would be invaluable. The feedback can include localization of the actual lesion to thus allow targeted tissue removal and more accurate removal of tissue surrounding the tumor. The feedback can also include a follow up of the effectiveness of treatment and post surgical appearance of potential additional lesions which would allow removal of them in a timely manner.

Embodiments of the present invention provide such a device. Moreover, such devices may be used with existing cystoscopy equipment and do not require any additional intravesical chemicals. The flexible fiber probe is simply inserted through the working channel of the cystoscope. The device of the invention can also be incorporated into a flexible cystoscope, which incorporates the optical device as described herein. For example, one can perform optical sampling of biopsies using such combination devices.

Therefore, embodiments of the present invention may be used for cystoscopy, uteroscopy, and percutaneous techniques. In addition, they may be used with open surgery, laparoscopic surgery, and robotic surgery. This technology fills the need for real-time instantaneous detection of cancer without the use and delay of histology. In addition, by coupling existing ablative or surgical technology, the device allows targeted treatment and spares majority of the normal tissue thus reducing side effects.

In general, one may use tissue samples to prepare a training set and a non-invasive real-time diagnostic and/or detection and/or treatment method for diseases, such as cancer in an organ system or a tissue. Accordingly, embodiments of the present invention may provide a non-invasive real-time method of detecting and/or treating cancer of a target tissue, comprising the steps of contacting the device with the target tissue, analyzing the output spectra, comparing the output spectra with a training set of spectra. The training set of spectra can comprise spectra obtained from normal tissue comparable with the diagnostic target, and various disease conditions affecting such tissue. When one compares the target tissue spectrum or spectra with the training set, one looks for an as near match as possible. This can be done visually or by using any known pattern recognition software. Once a match or a near match is obtained, one can then finalize the analysis and conclude, for example, that the target tissue is normal, if the spectra are a match with the normal tissue spectra. Similarly, cancer may be diagnosed if the spectra from the target tissue most closely match the training set spectra obtained from a tissue with cancer. In one embodiment, the training spectra are produced from age matched tissue samples. In another embodiment, an average spectra created from multiple samples having the same condition is used as the training spectrum.

Accordingly, in view of the foregoing, embodiments for conducting diagnosis of a tissue may comprise: a tissue manipulation apparatus; a multi-fiber optical probe with a tip adapted to be positioned at a site on the tissue and adapted to collect a diagnostic measurement from the tissue site, the multi-fiber optical probe comprising: at least one illumination fiber adapted to transmit photons into the tissue site; and at least one collection fiber adapted to collect a sample of the photons after the photons enter the tissue site, the sample of the photons providing the diagnostic measurement based on interaction of the photons with the tissue at the tissue site, wherein the tissue manipulation apparatus and the multi-fiber probe are positioned in an orientation that permits the device to operate on the same tissue at the tissue site. In some embodiments, the at least one illumination fiber is adapted to transmit photons into the tissue site while in contact with the tissue site, and the at least one collection fiber is adapted to collect a sample of the photons from the tissue site while in contact with the tissue site. Other embodiments further comprise a pulsed arc lamp transmitting photons to the at least one illumination fiber. In yet other embodiments, the at least one illumination fiber is adapted to transmit photons of a broadband light source. In one embodiment, the broadband light source has a wavelength range of 320 nanometers to 850 nanometers. In some embodiments, the at least one collection fiber transmits the sample of the photons to an analyzing spectrometer. In other embodiments, the at least one illumination fiber and the at least one collection fiber are separated by a fixed separation distance. In particular embodiments, the fixed separation distance is less than or equal to about 350 μm, center-to-center. Some embodiments further comprise a flexible elongate extension shaped to position the tissue manipulation apparatus and the multi-fiber optical probe at the tissue site. Particular embodiments comprise a channel within the flexible elongate extension, wherein the multi-fiber optical probe is positioned within the channel. In further embodiments, the tip of the multi-fiber optical probe is positioned proximate to the hinge of the two opposing jaws. In particular embodiments, the tip of the multi-fiber optical probe is extendible from the hinge of the two opposing jaws, whereby the multi-fiber optical probe extends to make contact with the tissue site. In other particular embodiments, the tip of the multi-fiber optical probe is fixed with respect to the hinge of the two opposing jaws, whereby the jaws grasp the tissue at the tissue site and bring the tissue into contact with the tip of the multi-fiber optical probe. In some embodiments, the tip of the multi-fiber optical probe is positioned at a base of the wire loop, whereby the tissue is brought into contact with the tip of the multi-fiber optical probe when the wire loop is tightened around the tissue at the tissue site. In other embodiments, the tissue manipulation apparatus is a device for cautery-ablation. In further embodiments, the sample of the photons providing the diagnostic measurement is based on scattering and absorption of the photons at the tissue site. In yet other embodiments, the device is disposable and includes connectors permitting removable connection to a pulsed arc lamp transmitting photons to the at least one illumination fiber and to a spectrometer receiving the scattered sample of photons collected by the at least one collection fiber.

In additional embodiments, a method for the diagnosis of a disease or disorder in a tissue site may comprise: contacting said tissue with the previous devices; and analyzing the diagnostic measurement from the device.

In other additional embodiments, a method for the simultaneous detection and treatment of a tissue may comprise: contacting a tissue with the previous devices; analyzing the diagnostic measurement received from the device; and treating the tissue with the tissue manipulation apparatus.

Further embodiments for conducting diagnosis of a tissue may comprise: a tissue manipulation apparatus; and a plurality of multi-fiber optical probes with tips adapted to be positioned at a plurality of tissue sites on the tissue to collect diagnostic measurements, each multi-fiber optical probe comprising: at least one illumination fiber adapted to transmit photons into one of the plurality of tissue sites; and at least one collection fiber adapted to collect a sample of the photons scattered after the photons enter the tissue site, the sample of the photons providing a diagnostic measurement based on interaction of the photons with the tissue at the tissue site, wherein at least one of the tips of the plurality of multi-fiber optical probes is positioned on the tissue manipulation apparatus. In some embodiments, a tip of one of the plurality of multi-fiber optical probes is positioned proximate to the hinge of the two opposing jaws. In other embodiments, the plurality of multi-fiber optical probes comprises a first multi-fiber optical probe with a tip positioned proximate to the hinge of the two opposing jaws, a second multi-fiber optical probe with a tip positioned at one of the two opposing jaws, and a third multi-fiber optical probe with a tip positioned at the other of the two opposing jaws. In yet other embodiments, the at least one illumination fiber of each of the plurality of multi-fiber optical probes is adapted to transmit photons into the tissue site while in contact with the tissue site, and wherein the at least one collection fiber of each of the plurality of multi-fiber optical probes is adapted to collect a sample of the photons from the tissue site while in contact with the tissue site.

Other embodiments for conducting diagnosis of a tissue may comprise: a reusable assembly comprising: a light source; and a spectrometer; and a plurality of disposable assemblies, each disposable assembly comprising: a tissue manipulation apparatus; and a multi-fiber optical probe adapted to collect a diagnostic measurement from a tissue site, the multi-fiber optical probe comprising: at least one illumination fiber adapted to transmit into the tissue site photons from the light source; and at least one collection fiber adapted to collect a sample of the photons after the photons enter the tissue site and transmit the sample of the photons to the spectrometer, the spectrometer providing a diagnostic measurement based on interaction of the photons with the tissue at the tissue site; wherein the disposable assembly is detachably connectable to one of the plurality of disposable assemblies at a time, and each of the disposable assemblies is detachably connected to the reusable assembly only a single time.

In further embodiments for conducting diagnosis of a tissue, a method may comprise: coupling a disposable assembly of to a reusable assembly into a diagnostic device; and applying the diagnostic device to a tissue site on a tissue; wherein the reusable assembly comprises: a light source; and a spectrometer; and one takes an optical measurement of a suspected tissue site, analyzes said measurements in real time and determines whether to use the tissue manipulation apparatus on said measured tissue site.

While the present invention has been described in connection with a number of exemplary embodiments, and implementations, the present inventions are not so limited, but rather cover various modifications, and equivalent arrangements, which fall within the purview of prospective claims.

All the references cited throughout the specification are herein incorporated by reference in their entirety.

1. A device for conducting diagnosis of a tissue, the device comprising:
a tissue manipulation apparatus; and
a multi-fiber optical probe with a tip adapted to be positioned at a site on the tissue and adapted to collect a diagnostic measurement from the tissue site, the multi-fiber optical probe comprising:
at least one illumination fiber adapted to transmit photons into the tissue site; and
at least one collection fiber adapted to collect a sample of the photons after the photons enter the tissue site, the sample of the photons providing the diagnostic measurement based on interaction of the photons with the tissue at the tissue site,
wherein the tissue manipulation apparatus and the multi-fiber probe are positioned in an orientation that permits the device to operate on the same tissue at the tissue site.

2. The device according to claim 1, wherein the at least one illumination fiber is adapted to transmit photons into the tissue site while in contact with the tissue site, and wherein the at least one collection fiber is adapted to collect a sample of the photons from the tissue site while in contact with the tissue site.

3. The device of claim 1, wherein the device is disposable and includes couplers permitting removable connection to a pulsed arc lamp transmitting photons to the at least one illumination fiber and to a spectrometer receiving the scattered sample of photons collected by the at least one collection fiber.

4. The device according to any of claims 1, 2 or 3, wherein a broadband light source is used and has a wavelength range of 320 nanometers to 850 nanometers.

5. The device according to any of claims 1, 2 or 3 further comprising a flexible elongate extension shaped to position the tissue manipulation apparatus and the multi-fiber optical probe at the tissue site.

6. The device according to any of claims 1, 2 or 3, wherein the tissue manipulation apparatus is a biopsy forceps with two opposing jaws joined at a hinge, or a biopsy snare comprising a wire loop adapted to tighten around the tissue at the tissue site.

7. A device for conducting diagnosis of a tissue, the device comprising:
a tissue manipulation apparatus; and
a plurality of multi-fiber optical probes with tips adapted to be positioned at a plurality of tissue sites on the tissue to collect diagnostic measurements, each multi-fiber optical probe comprising:
at least one illumination fiber adapted to transmit photons into one of the plurality of tissue sites; and
at least one collection fiber adapted to collect a sample of the photons scattered after the photons enter the tissue site, the sample of the photons providing a diagnostic measurement based on interaction of the photons with the tissue at the tissue site,
wherein at least one of the tips of the plurality of multi-fiber optical probes is positioned on the tissue manipulation apparatus.

8. The device according to claim 7, wherein the tissue manipulation apparatus is a biopsy forceps with two opposing jaws joined at a hinge.

9. A system for conducting diagnosis of a tissue, the system comprising:
a reusable assembly comprising:
a light source; and
a spectrometer; and
a plurality of disposable assemblies, each disposable assembly comprising:
a tissue manipulation apparatus; and
a multi-fiber optical probe adapted to collect a diagnostic measurement from a tissue site, the multi-fiber optical probe comprising:
at least one illumination fiber adapted to transmit into the tissue site photons from the light source; and
at least one collection fiber adapted to collect a sample of the photons after the photons enter the tissue site and transmit the sample of the photons to the spectrometer, the spectrometer providing a diagnostic measurement based on interaction of the photons with the tissue at the tissue site;
wherein the disposable assembly is detachably connectable to one of the plurality of disposable assemblies at a time, and each of the disposable assemblies is detachably connected to the reusable assembly only a single time.

10. A method for conducting diagnosis of a tissue, wherein the method comprises:
coupling the disposable assembly of claim 3 to a reusable assembly into a diagnostic device; and
applying the diagnostic device to a tissue site on a tissue;
wherein the reusable assembly comprises:
a light source; and
a spectrometer; and
one takes an optical measurement of a suspected tissue site, analyzes said measurements in real time and determines whether to use the tissue manipulation apparatus on said measured tissue site.
